(19) United States
(12) Patent Application Publication
(10) Pub. No.: US 2008/0261263 A1
(43) Pub. Date: Oct. 23, 2008

(54) In Vitro Evaluation of Micro-Organisms and Their Antimicrobial Agent Susceptibilities

(75) Inventors: Randall Alexander Allardyce, North Canterbury (NZ); Vaughan Stephen Langford, Christchurch (NZ); Jennifer Margaret Scotter, North Canterbury (NZ); David Roger Murdoch, Christchurch (NZ)

Correspondence Address: D. PETER HOCHBERG CO. L.P.A., 1940 EAST 6TH STREET, CLEVELAND, OH, 44114, US

(73) Assignee: SYFT Technologies Limited, Middleton, Christchurch (NZ)

(21) Appl. No.: 11/884,410

(22) PCT Filed: Feb. 14, 2006
(86) PCT No.: PCT/NZ06/00019

§ 371 Date: Aug. 15, 2007

(30) Foreign Application Priority Data
Feb. 15, 2005 (NZ) 538235

(51) Int. Cl. C12Q 1/18

(52) U.S. Cl. 435/32

(57) ABSTRACT

A method of identifying a micro-organism and determining the susceptibility of the micro-organism to an antimicrobial agent by inserting a sample of the micro-organism in a primary culture container and allowing the micro-organism to multiply. The primary culture is then divided into a plurality of secondary cultures and the metabolic volatile or semi-volatile compounds (VCs) in the headspace above the cultures are analysed by SIFT-MS to ascertain whether micro-organisms exist in the culture and determine the type of micro-organism. The secondary cultures are then divided into a number of tertiary cultures and an antimicrobial agent is introduced whereupon the VCs in the headspace above the tertiary cultures are analysed by SIFT-MS to determine the susceptibility of the micro-organism to the antimicrobial agent at various concentrations of the antimicrobial agent.

This application is a National Stage application of International Application No. PCT/NZ2006/000019, filed on Feb. 14, 2006, which claims priority of New Zealand application No. 538235 filed on Feb. 15, 2005.

### BACKGROUND OF THE INVENTION

1. Field of the Invention

This invention relates to determining the metabolic consequences of an antimicrobial agent for micro-organisms, and in particular, to identifying a micro-organism and the foregoing metabolic sequences; and in particular to utilizing SIFT-MS to detect and identify microbial growth in cultures, the in vitro determination of the susceptibility of micro-organisms to antimicrobial agents and the utilization of SIFT-MS to monitor the growth of specific micro-organisms.

2. Description of the Prior Art

It is known to use SIFT-MS to detect and identify micro-organisms by measuring their production of metabolic volatile or semi-volatile compounds (VCs). SIFT-MS combines chemical ionization of analyte VCs using H₃O+, NO+ or O₂+ with fast flow tube quadrupole-mediated identification and quantification in complex mixtures such as breath and ambient air regardless of the water vapor content in (near) real time. Product ions can then be identified with reference to a molecular-ion reaction and rate coefficient database.

It is also known that serious infections as well as other diseases are associated with recognizable odours—Labarca J A, Pegues D A, Wagar E A, Hindler J A, Bruckner D A. Something's rotten: a nosocomial outbreak of malodorous Pseudomonas aeruginosa. Clin Infect Dis 1998; 26:1440-1446.

Bacterial infections are typically diagnosed by culturing samples of tissues or body fluids including blood. A common process is to use BactT/ALERT® bottles which contain small amounts of culture medium. The process of growing the micro-organisms can be monitored through colour change and/or production of CO₂. This system is fairly simple but lacks specificity and tends to be slow with typical times to obtain definitive positive or negative being 12-48 hours.

In vitro bacterial culture studies using gas chromatography mass spectrometry have also identified a large number of metabolic volatile compounds including fatty acids, aliphatic alcohols, ketones, dimethyl polysulphides and alkenes.

It is known that certain bacteria give off signature profiles of VCs which can be used to identify the bacteria. Gas chromatography mass spectrometry has also been used to identify these profiles.

Prior to the present invention, techniques used to identify whether or not micro-organisms are present in blood samples takes between 12 to 48 hours and this presents serious problems in early diagnosis, particularly since the identification is not specific as to the type of micro-organism. A further disadvantage with current techniques is that the identification of the bacteria gives limited indication of the effect of an antibiotic on the bacteria, nor of the type of antibiotic that will be most effective against the bacteria.

Larsson et al in 1978 compared the Gas Chromatography—Flame Ionisation Detection (GC-FID) and Gas Chromatography—Mass Spectrometry (GC-MS) detected volatile headspace metabolic products of cultured anaerobic species with complete liquid culture medium and solvent extracts. —Larsson L, Mardh P A, Odham G. Analysis of amines and other bacterial products by head-space gas chromatography. Acto Pathol Microbiol Scand [B]. 1978; 86:207-213. It was shown that volatile fatty acids could be detected in all three sources, whereas alcohols were detected only in the headspace chromatograms—Larsson L, Mardh P A, Odham G. Detection of alcohols and volatile fatty acids by head-space gas chromatography in identification of anaerobic bacteria. J Clin Microbiol 1978; 7:23-27.

In 1982 Larsson et al demonstrated that automated heated anaerobe culture headspace GC injection using a fused silica capillary column provided more diagnostic information on volatile alcohols and fatty acids than GC using ether extracts and packed columns. —Larsson L, Holst E. Feasibility of automated head-space gas chromatography in identification of anaerobic bacteria Acta Pathol Microbiol Immunol Scand [B]. 1982; 90:125-130.

In 1998 Kiviranta et al demonstrated that the qualitative identification of three bacterial and two fungal species from culture headspace volatile organic compounds adsorbed on Tenax TA and analysed by GC-MS was highly dependent on the culture medium and the species—Kiviranta H, Tuomainen A, Reiman M, Laitinen S, Liesivuori J Nevalainen A. Qualitative identification of volatile metabolites from two fungi and three bacteria species cultivated on two media. Cent Eur J Public Health 1998; 6:296-299.

A variety of solid phase micro extraction (SPME) materials has also been used with GC-MS to profile fungal volatile metabolites—Wady L, Bunte A, Pehrson C, Larsson L. Use of gas chromatography-mass spectrometry/solid phase microextraction for the identification of MVCx from moldy materials. J Microbiol Methods. 2003; 52:325-332, and also Jelen H H. Use of solid phase microextraction (SPME) for profiling fungal volatile metabolities. Lett Appl Microbiol 2003; 36:263-267.

Further SPME materials have also been used with GC-MS to profile bacterial volatile metabolites.

These studies found that all the fibers showed varied and significantly different efficiency and selectivity, and that the VC profiles were highly dependent on the extraction method and the composition of the culture medium. Julak et al concluded that this limits the accurate characterization of particular bacterial species from different clinical samples—Julak J, Prochazkova-Fancisci E, Stranska E, Rosova V. Evaluation of exudates by solid phase microextration-gas chromatography. J Microbiol Methods 2003; 52:115-122.

A number of analytical methods have also been used for detection and identification of bacteria, including, gas chromatographic determination of volatile fatty acids (VFA) and non-volatile (NVFA) carboxylic acids profiles. The pattern of the fermentative metabolism end products in spent culture media is of great importance in the identification of pure cultures of anaerobic bacteria, to a lesser extent in facultative anaerobic bacteria. These profiles are, in defined conditions, more or less characteristic of bacterial species. However, they are also more or less dependent on the composition of the used cultivation medium, and the straight analysis of clinical body fluids, which are poorly defined ‘media’, is thus somewhat limited. Nevertheless, the analyses of blood cultures and other fluids have been reported.

Julak and colleagues attempted to verify the simple and rapid chromatographic determination of VFA in blood cultures, which may improve the detection of anaerobic infections, which may be life-threatening but often not detected by routine microbiological examination. Julak J, Stranska E, Prochazdova-Francisci E, Rosova V. Blood cultures evaluation by gas chromatography of volatile fatty acids. Med. Sci. Monit. 2000; 6:605-610.

Wang T, et al have recently expanded their SIFT-MS database to include the kinetic data for the reactions of several compounds related to the emissions produced by Pseudomonas species in vitro. Wang T, Smith D, Spanel P. Selected ion flow tube SIFT studies of the reactions of H₃O+, NO+ and O₂+ with compounds released by Pseudomonas and related bacteria. Intl J Mass Spectrometry 2004; 233:245-251 This article also includes a report on VCs produced by Pseudomonas species of bacteria.

Electronic noses utilizing a variety of data analysis techniques have also demonstrated their ability to discriminate between groups of bacterial species and urine infected with Escherichia coli or Staphylococcus species—Pavlou A, Turner A P, Magan N Recognition of anaerobic bacterial isolates in vitro using electronic nose technology. Lett Appl Microbiol 2002; 35:366-369 and also Pavlou A K, Magan N. McNulty C, Jones J, Sharp D, Brown J, Turner A P. Use of an electronic nose system for diagnoses of urinary tract infections. Biosens Bioelectron 2002; 17:893-899.

Another approach to bacterial identification has involved the thermal desorption of whole bacterial cells using ion mobility spectrometry (IMS). Although prior sample clean-up and concentration steps are not required, access to microgram quantities of purified micro-organisms is. Vinopal R T, Jadamec J R, deFur P, Demars A L, Jakubielski S. Green C, Anderson C P, Dugas J E, DeBono R F. Fingerprinting bacterial strains using ion mobility spectrometry. Analytica Chimica Acta 2002; 21745:1-13.

Shnayderman et al describe the use of micromachined differential (ion) mobility spectrometry to measure headspace gases from bacteria growing in liquid culture. They applied pattern discovery/recognition algorithms (ProteomeQuest) to identify the VC profiles of four species including Escherichia coli, Bacillus subtilis, Bacillus thuringiensis and Mycobacterium smegmatis. They conclude that their combination of technology and bioinformatics data analysis has potential for diagnosis of bacterial infections. Shnayderman M, Mansfield B, Yip P, Clark H A, Krebs M D, Cohen S J, Zeskind J E, Ryan E T, Dorkin H L, Callahan M V, Stair T O, Gelfand J A, Gill C J, Hitt B, Davis C E. Species-specific bacteria identification using differential mobility spectrometry and bioinformatics pattern recognition. Anal Chem. 2005; 77(18):5930-7.

Lechner et al used proton transfer reaction mass spectrometry (PTR-MS) to measure the liquid culture headspace VCs of Escherichia coli, Klebsiella, Citrobacter, Pseudomonas aeruginosa, Staphylococcus aureus and Helicobacter pylori. The patterns of VCs detected differed in quantity and composition for each species tested. The authors erroneously conclude that they were the first to describe the headspace screening of bacterial cultures as a potential microbiological diagnostic approach. Lechner M, Fille M, Hausdorfer J, Dierich M P, Rieder J Diagnosis of bacteria in vitro by mass spectrometric fingerprinting: a pilot study. Curr Microbiol. 2005; 51(4):267-9. Electronic publication Jul. 27, 2005.

Of considerable significance to the invention is prior art relating to the detection of volatile compounds from fungi grown on a variety of laboratory media by SIFT-MS, by Scotter et al. The fungi examined in this study were Aspergillus flavus, Aspergillus fumigatus, Mucor racemosus, Fusarium solani and Cryptococcus neoformans grown on or in malt extract agar, Columbia agar, Sabouraud's dextrose agar, blood agar and brain-heart infusion broth. (Scotter J M, Langford V S, Wilson P F, McEwan M J, Chambers S T, Real time detection of common microbial volatile organic compounds from medically important fungi by Selected Ion Flow Tube-Mass Spectrometry (SIFT-MS). Journal of Microbiological Methods 2005; 63:127-34).

Common metabolites (ethanol, methanol, acetone, acetaldehyde, methanethiol and crotonaldehyde) were detected and quantified. They found the presence and quantity of volatiles produced were strongly dependent on the culture medium, but concluded that those fingerprints had potential for use in in vitro diagnostic tests to form the basis for species specific identification of medically important fungi.

It is recognized that it would be of great clinical value, if real-time, non invasive measurements of breath or the headspaces above urine, faeces, blood or sputum could replace the sample preparation and discrimination against reactive or low molecular weight molecules experienced with GC-MS.

In particular it would be of considerable benefit if the antimicrobial susceptibilities of micro-organisms, such as but not limited to, bacteria, viruses, fungi, parasites and single celled protists could be rapidly identified from routine test sample headspaces.

### SUMMARY OF THE INVENTION

It is therefore an object of this invention to utilise SIFT-MS to rapidly detect and identify microbial growth in cultures.

It is a still further object to be able to rapidly and effectively determine the susceptibility of micro-organisms to antimicrobial agents in vitro.

It is a yet further object of the invention to utilise SIFT-MS to identify characteristic trace gases and to utilise such measurements to monitor in vitro or in vivo growth of specific micro-organisms.

In one form the invention relates to a method of determining the metabolic consequences of an antimicrobial agent for a micro-organism comprising

establishing the presence of a micro-organism(s) by sampling by means of SIFT-MS the VCs produced by the bacterium or micro-organism,

identifying the micro-organism(s)

analysing the VCs produced by the micro-organism(s) and

determining the antimicrobial susceptibility of the micro-organism(s).

The invention also relates to a method of identifying a micro-organism(s) and the metabolic consequences of an antimicrobial agent on the micro-organism comprising the steps of

inserting a sample containing the micro-organism(s) into a primary culture container,

allowing micro-organism(s) within the primary culture of the sample to multiply in the container

dividing the primary culture into a plurality of secondary cultures and charging separate containers with individual secondary cultures,

analysing the VCs in the headspace above the secondary cultures by means of SIFT-MS to ascertain whether micro-organisms exist in the secondary culture

determining the type of micro-organism,

splitting the secondary cultures into a plurality of tertiary cultures and inserting the tertiary cultures into separate containers with each container including a specific antimicrobial agent at a specific concentration,

analysing the VCs in the headspace above each tertiary culture to determine the antimicrobial susceptibility of the micro-organism.

Preferably the primary culture is divided into the secondary cultures after a period of rest or incubation from the commencement of the test.

Preferably the primary culture is divided into a plurality of secondary cultures with one of the secondary cultures being retained as a control culture to enable a positive or negative report to be generated as to the presence of an infectious bacterium or micro-organism and to enable the infection to be identified.

Preferably the primary culture is divided into three secondary cultures.

Preferably the secondary culture is tested after a period of rest from the commencement of the test and if the test provides a positive result, the secondary culture is divided into the plurality of tertiary cultures.

Preferably the secondary culture is divided into a plurality of tertiary cultures which are each combined with a specific antibiotic or antimicrobial substance at a specific concentration.

Preferably the tertiary cultures are tested after a period of rest from the commencement of the test to determine the susceptibility of the bacterium or micro-organism to the antibiotic or antimicrobial substance.

Preferably the susceptibility of the bacterium or micro-organism to the antimicrobial agent is determined by the presence of either a high concentration of the chosen antibiotic or antimicrobial substance or a medium concentration of the antibiotic or antimicrobial substance.

In another aspect the invention relates to a method of identifying an infectious micro-organism and the metabolic consequences of an antimicrobial agent in a blood culture comprising the steps of

inserting a primary blood culture into a container,

allowing bacteria or organisms within the primary blood culture to multiply in the container

dividing the culture into a plurality of secondary cultures and charging separate containers with individual secondary cultures,

analysing the VCs in the headspace above the secondary cultures by means of SIFT-MS to ascertain whether an micro-organism(s) exists in the secondary blood culture and to determine the type of micro-organism,

splitting the secondary cultures into a plurality of tertiary cultures and inserting the tertiary cultures into separate containers with each container including a specific antimicrobial agent at a specific concentration,

analysing the VCs in the head space above each tertiary culture to determine whether the micro-organism in the tertiary culture has grown and to ascertain whether the antimicrobial agent has been inhibitory to the growth of the micro-organism.

Preferably a report is generated in which the micro-organism is identified and the antimicrobial susceptibility of the micro-organism is displayed.

### DETAILED DESCRIPTION OF THE PREFERRED EMBODIMENTS

SIFT-MS involves the generation of precursor ions (e.g. H₃O+, O₂+, NO+) from a discharge source that are mass selected by an “upstream” quadrupole mass filter. The selected ion species is then injected into the flow tube by a fast flowing stream of inert carrier gas, eg helium.

In one form of the method headspace atmospheres are introduced above conventional Biomerieux BacT/ALERT® aerobic or anaerobic blood cultures at a controlled rate into the flow tube precursor ion stream. The count rates of the resulting product ions are then to be determined by a “downstream” quadrupole and particle multiplier detector. Operating in full scan mode, the detector quadrupole is scanned over a predetermined m/z range to obtain a spectrum of product ions. In selected ion mode (SIMscan) the count rate of selected product ions is determined as the downstream spectrophotometer is switched to and remains on selected m/zs.

In the experiments to test whether it is possible to identify specific micro-organisms by SIFT-MS, full scans were used to compare test product ion spectra against those of appropriate controls, while SIM scans were used to determine the count rates of selected test product ions of interest.

Bacterial cultures were studied as follows:

| Species | Type strain |
|---|---|
| Streptococcus pneumoniae | ATCC 49619 |
| Pseudomonas aeruginosa | ATCC 27853 |
| Escherichia coli | ATCC 25922 |
| Staphylococcus aureus | ATCC 25923 |
| Neisseria meningitides | NZESR 1033 |

### Bacterial Blood Culture

Standard Biomerieux (Durham, N.C., USA) BacT/ALERT® SA, FA and SN disposable, plastic culture bottles containing 40 ml of culture medium were used throughout the experiments. 9 ml of healthy, uninfected human blood and 1 ml aliquots of antibiotics prepared in sterile water were added (in the relevant experiments) as required to each bottle prior to the addition of 1 ml of bacterial species suspended in sterile physiological saline.

### Full Mass Scan Bacterial VC Identification

Less than 10 colony forming units (CFU) of each test species were inoculated into Biomerieux BacT/ALERT® SA blood culture bottles including 9 ml of fresh uninfected blood. VCs from triplicate cultures of five bacterial species were compared with triplicate sterile blood (only) BacT/ALERT® SA controls. Bacterial-specific VCs were identified from full mass scans (all three ion precursors) after 24 hours of incubation at 37° C.

### Selected Ion Mode (SIMscan) VC Measurements

SIFT-MS mass scan studies comparing 24 hour BacT/ALERT®SA blood culture headspace VCs with medium+blood controls identified a test panel of SIM VC analytes suitable for the five bacterial species tested. These bacterial metabolites included acetaldehyde, acetic acid, acetone, 2-aminoacetophenone, ammonia, dimethyldisulfide (DMDS), dimethylsulfide (DMS), formaldehyde, ethanol, hydrogen sulfide, indole, methanethiol, pentanols and propanol. Seven absolute concentration measurements in parts per billion (ppb v/v) were integrated, averaged and recorded separately for acetaldehyde, acetic acid, ethanol, acetone, ammonia, hydrogen sulfide, DMS and DMDS during each 30 second SIMscan for each triplicate culture of the five test species after incubation in BacT/ALERT aerobic media. The results are recorded in Table 1 (below).

TABLE 1
Bacterial VCs at 6 hours in BacT/ALERT ® FA aerobic medium

| | Mean analyte VC concentration & range (parts per billion v/v) | | | | | | | | |
|---|---|---|---|---|---|---|---|---|---|
| | | | | | | | Methane- | | |
| 6 hour culture | Acetaldehyde | Acetic Acid | Ethanol | Acetone | Ammonia | H₂S | thiol | DMS | DMDS |
| Control | 890 | 6900 | 1200 | 3600 | 1000 | 10 | ND^{a} | 690 | 200 |
| medium + | (810-940) | (6850-6980) | (1100-1240) | (3400-3750) | (890-1100) | (10-10) | | (630-750) | (180-220) |
| Blood | | | | | | | | | |
| Pseudomonas | 1100 | 16000 | 1500 | 6600 | 1100 | 40 | ND | 860 | 330 |
| aeruginosa | (1000-1200) | (15850-16200) | (1390-1600) | (6260-6950) | (950-1200) | (30-50) | | (800-960) | (300-350) |
| Streptococcus | 5300 | 460 | 3000 | 5100 | 370 | 20 | 60 | 4300 | 180 |
| pneumoniae | (5220-5400) | (440-490) | 2880-3160) | (4950-5280) | (350-380) | (20-20) | (40-70) | (3990-4500) | (160-190) |
| Escherichia | 11000 | 5400 | 21000 | 6100 | 500 | 4100 | 750 | 9600 | 430 |
| coli | (10900-11900) | (5290-5500) | (19400-23200) | (6000-6300) | (470-550) | (4000-4280) | (650-820) | (9350-10400) | (410-450) |
| Staphylococcus | 2400 | 1200 | 5800 | 3500 | 1800 | 60 | 180 | 2200 | 280 |
| aureus | (2360-2440) | (1090-1300) | (5500-6300) | (3170-3810) | (1690-1880) | (50-70) | (170-190) | (2100-2360) | (260-310) |
| Neisseria | 350 | 870 | 770 | 7900 | 1200 | ND | ND | 320 | 360 |
| meningitidis | (300-380) | (790-940) | (670-870) | (6980-8550) | (1080-1330) | | | (300-330) | (340-390) |

ND = analyte not detected

In the second series of experiments, the times taken to achieve a positive result by SIFT-MS were directly compared to blood culture bottles were incubated at 37° C. with agitation on the automated BacT/ALERT® 3D blood culture system. Triplicate replica sets of each strain at each plate count-confirmed dilution (Table 3) were incubated; the first set remained within the BacT/ALERT® system until a positive result was recorded, the second set was tested by SIFT-MS at 8 hours incubation, and the third set tested by SIFT-MS at 24 hours (Table 4). Any bottle intended for testing by SIFT-MS, that prior to testing at the given time point (ie 8 or 24 hours) returned a positive result by the conventional system, was retained under incubation conditions and tested by SIFT-MS at the planned time point. The conventional time to positive result (TTP) was recorded as a decimal fraction of 24 hours by the automated BacT/ALERT® 3D blood culture machine. Results were tabulated and converted into time in hours and minutes, and recorded for each bottle. Negative control bottles, consisting of 10 ml sterile blood only, were incubated for 8 and 24 hours prior to testing by SIFT-MS, and those intended for conventional blood culture were left to incubate for 3 days, after which the result was accepted as negative, or no bacterial growth. The results for aerobic and anaerobic cultures are presented in tables 4, 5, 6, and 7.

### Analysis

Time to positive (TTP) result was recorded for all bottles remaining on the blood culture system. Bottles intended for SIFT-MS analysis that flagged positive prior to the projected testing time were removed from the machine, and incubation continued without agitating at 37° C. The mean concentration of each VC metabolite listed above was measured for triplicate negative controls as for other cultures. The standard deviation for each analyte for negative controls at each assay time (ie 8 and 24 hours) was calculated. A threshold, or negative cutoff value for each analyte was determined as the mean of the negative control values plus 2 standard deviations from that mean. A bottle was recorded as positive if a level above the threshold was measured for at least one analyte (Tables 4 and 5).

Each culture, detailed above, was evaluated for statistically significant production of VCs. The mean concentration for each VC from each group of three bottles were tested with a two-tailed student's T-test for unmatched samples, against the appropriate mean VC concentration for each corresponding negative control group. Samples returning p values of ≦0.05 were considered statistically significant (Tables 6 and 7).

### Results

### Direct Comparison of SIFT-MS and Conventional Blood Culture Time to Positive Result

Plate counts confirming bacterial inoculations are recorded in Table 2. A total of 198 bottles were tested according to the schedule shown in Table 3. Plate counts confirming bacterial inoculations are recorded in Table 3. Results are shown in Tables 4 and 5. In summary, none of the negative control bottles returned a positive result by either system, indicating that no contamination was present in the experimental system, and that the thresholds, or negative cutoff calculations for SIFT-MS were appropriate. In general, the triplicate samples were well clustered, both for TTP for conventional blood culture, and absolute concentrations of analytes by SIFT-MS.

TABLE 2
Final plate-count confirmed dilutions for time to positive result
bacterial blood cultures.

| | +O₂ | | | −O₂ | | |
|---|---|---|---|---|---|---|
| Organism | “100 CFU” | “5 CFU” | | “100 CFU” | “5 CFU” | |
| S. pneumoniae | 28 CFU | 1-2 | CFU | 28 CFU | 1-2 | CFU |
| Ps. aeruginosa | 7 CFU | 0-1 | CFU | 64 CFU | 3 | CFU |
| E. coli | 7 CFU | 0-1 | CFU | 82 CFU | 4 | CFU |
| S. aureus | 44 CFU | 2 | CFU | 55 CFU | 3 | CFU |

| | | | | | |
|---|---|---|---|---|---|
| N. meningitidis | 4 CFU | — | 13 CFU | 1 | CFU |

TABLE 3
Bacterial blood culture time to positive versus VC evaluation scheme

| | | | Initial | |
|---|---|---|---|---|
| | | | inoculum | Analysis point |
| Bottle # | Organism | Test system | (CFU) | (culture time) |
| 1-3 | S. pneumoniae | conventional. | 10² | Until positive |
| 4-6 | S. pneumoniae | conventional. | 5 | Until positive |
| 7-9 | S. pneumoniae | SIFT-MS | 10² | 8 hrs |
| 5-12 | S. pneumoniae | SIFT-MS | 5 | 8 hrs |
| 13-15 | S. pneumoniae | SIFT-MS | 10² | 24 hrs |
| 16-18 | S. pneumoniae | SIFT-MS | 5 | 24 hrs |
| 19-21 | Ps. aeruginosa | conventional. | 10² | Until positive |
| 22-24 | Ps. aeruginosa | conventional. | 5 | Until positive |
| 25-27 | Ps. aeruginosa | SIFT-MS | 10² | 8 hrs |
| 28-30 | Ps. aeruginosa | SIFT-MS | 5 | 8 hrs |
| 31-33 | Ps. aeruginosa | SIFT-MS | 10² | 24 hrs |
| 34-36 | Ps. aeruginosa | SIFT-MS | 5 | 24 hrs |
| 37-39 | E. coli | conventional. | 10² | Until positive |
| 40-42 | E. coli | conventional. | 5 | Until positive |
| 43-45 | E. coli | SIFT-MS | 10² | 8 hrs |
| 46-48 | E. coli | SIFT-MS | 5 | 8 hrs |
| 49-51 | E. coli | SIFT-MS | 10² | 24 hrs |
| 52-54 | E. coli | SIFT-MS | 5 | 24 hrs |
| 55-57 | S. aureus | conventional. | 10² | Until positive |
| 58-60 | S. aureus | conventional. | 5 | Until positive |
| 61-63 | S. aureus | SIFT-MS | 10² | 8 hrs |
| 64-66 | S. aureus | SIFT-MS | 5 | 8 hrs |
| 67-69 | S. aureus | SIFT-MS | 10² | 24 hrs |
| 70-72 | S. aureus | SIFT-MS | 5 | 24 hrs |
| 73-75 | N. meningitidis | conventional. | 10² | Until positive |
| 76-78 | N. meningitidis | conventional. | 5 | Until positive |
| 79-81 | N. meningitidis | SIFT-MS | 10² | 8 hrs |
| 82-84 | N. meningitidis | SIFT-MS | 5 | 8 hrs |
| 85-87 | N. meningitidis | SIFT-MS | 10² | 24 hrs |
| 88-90 | N. meningitidis | SIFT-MS | 5 | 24 hrs |
| 91-93 | Neg. control | conventional. | 0 | Until last positive |
| 94-96 | Neg. control | SIFT-MS | 0 | 8 hrs |
| 97-99 | Neg. control | SIFT-MS | 0 | 24 hrs |

TABLE 4
Conventional blood culture vs SIFT-MS-time to positive
result-Aerobic.

| | | | | SIFT-MS | |
|---|---|---|---|---|---|
| | | Blood culture | | (no. pos/total) | |
| Organism | Inoc | TTP | no. pos/total | 8 hrs | 24 hrs |
| S. pneumoniae | 100 | 15 hr 0 min | 3/3 | 3/3 | 3/3 |
| S. pneumoniae | 5 | 16 hr 54 min | 3/3 | 3/3 | 3/3 |
| Ps. aeruginosa | 100 | 18 hr 35 min | 3/3 | 3/3 | 1/3 |
| Ps. aeruginosa | 5 | 19 hr 55 min | 1/3 | 3/3 | 3/3 |
| E. coli | 100 | 14 hr 17 min | 3/3 | 3/3 | 3/3 |
| E. coli | 5 | 14 hr 44 min | 2/3 | 3/3 | 3/3 |
| S. aureus | 100 | 14 hr 38 min | 3/3 | 3/3 | 3/3 |
| S. aureus | 5 | 15 hr 29 min | 3/3 | 3/3 | 3/3 |
| N. meningitidis | 100 | 19 hr 12 min | 3/3 | 2/3 | 3/3 |
| N. meningitidis | 5 | 20 hr 13 min | 3/3 | 1/3 | 3/3 |

TABLE 5
Conventional blood culture vs SIFT-MS-time to positive
result-Anaerobic.

| | | Blood culture | | SIFT-MS | |
|---|---|---|---|---|---|
| Organism | Inoc | TTP | no. pos/total | 8 hrs | 24 hr |
| S. pneumoniae | 100 | 14 hr 9 min | 3/3 | 3/3 | 3/3 |
| S. pneumoniae | 5 | 15 hr 21 min | 3/3 | 3/3 | 3/3 |
| Ps. aeruginosa | 100 | 18 hr 43 min | 3/3 | 3/3 | 3/3 |
| Ps. aeruginosa | 5 | 20 hr 38 min | 3/3 | 3/3 | 3/3 |
| E. coli | 100 | 12 hr | 3/3 | 3/3 | 3/3 |
| E. coli | 5 | 13 hr 2 min | 2/3 | 3/3 | 3/3 |
| S. aureus | 100 | 13 hr 45 min | 3/3 | 3/3 | 3/3 |
| S. aureus | 5 | 15 hr 28 min | 3/3 | 3/3 | 3/3 |
| N. meningitidis | 100 | 21 hr | 2/3 | 2/3 | 3/3 |
| N. meningitidis | 5 | 21 hr | 2/3 | 0/3 | 3/3 |

Positive results (Table 6) were returned by SIFT-MS at 8 hours for all organisms, under aerobic and anaerobic conditions, with the exception of N. meningitidis, one bottle at 10² CFU, and two bottles at 5 CFU, under aerobic growth conditions, and the same organism under anaerobic growth conditions, with one bottle at 10² CFU and all three bottles at 5 CFU failing to return a positive result.

TABLE 6
VCs produced at statistically significant levels at 8 hours by five
bacteria under aerobic and anaerobic growth conditions

| | | Mean | | | Mean | |
|---|---|---|---|---|---|---|
| Organism | VC | (ppb) | p | VC | (ppb) | p |
| | 8 hours 100 CFU | | 8 hours 5 CFU | | | |
| S. pneumoniae | acetaldehyde | 1578 | 0.003 | Acetaldehyde | 1329 | 0.002 |
| | Acetic acid | 1417 | 0.003 | Acetic acid | 932 | 0.003 |
| | Ethanol | 3168 | 0.007 | Ethanol | 2255 | 0.04 |
| | Pentanols | 212 | 0.024 | Pentanols | 162 | 0.03 |
| | Acetone | 3773 | 0.016 | Acetone | 3034 | 0.001 |
| | Ammonia | 746 | 0.021 | Ammonia | 693 | 0.013 |
| | Hydrogen | 33 | 0.01 | | | |
| | Sulphide | | | | | |
| | Dimethyl | 45 | 0.03 | | | |
| | sulphide | | | | | |
| | Dimethyl disulphide | 472 | 0.012 | Dimethyl disulphide | 281 | 0.01 |
| | Trimethylamine | 193 | 0.034 | | | |
| | Indole | 55 | 0.03 | | | |
| | Propene | 2916 | 0.006 | 2 amino | 58 | 0.013 |
| | | | | acetophenone | | |
| | Mass 101 | 66 | 0.006 | Propene | 2742 | 0.018 |
| | Mass 105 | 198 | 0.04 | Mass 105 | 166 | 0.036 |
| | Mass 139 | 105 | 0.004 | Mass 121 | 163 | 0.017 |
| Ps. aeruginosa | Ammonia | 322 | 0.017 | ammonia | 307 | 0.011 |
| E. coli | Acetic acid | 464 | 0.045 | Ammonia | 282 | 0.004 |
| | Ethanol | 1988 | 0.04 | Methanethiol | 12 | 0.034 |
| S. aureus | Ammonia | 199 | 0.033 | Ammonia | 204 | 0.032 |
| | Dimethylsulphide | 75 | 0.015 | | | |
| N. meningitidis | — | | | — | | |
| | 8 hrs 100 CFU | | 8 hrs 5 CFU | | | |
| Anaerobic | | | | | | |
| S. pneumoniae | Acetone | 752 | 0.03 | Ammonia | 883 | 0.011 |
| Ps. aeruginosa | Acetone | 792 | 0.014 | Acetone | 768 | 0.007 |
| | Dimethylsulphide | 26 | 0.022 | Dimethylsulphide | 23 | 0.049 |
| | Indole | 29 | 0.011 | | | |
| | Propene | 817 | 0.024 | | | |
| E. coli | Acetaldehyde | 588 | 0.001 | | | |
| | Ammonia | 632 | 0.041 | | | |
| | Mass 139 | 104 | 0.033 | | | |
| S. aureus | Acetaldehyde | 549 | 0.012 | Acetaldehyde | 503 | 0.024 |
| | Dimethylsulphide | 10 | 0.035 | | | |
| N. meningitidis | — | | | Propene | 1400 | 0.035 |

At 24 hours (Table 7), all bottles returned positive results under aerobic and anaerobic growth conditions, with the exception of Ps. aeruginosa, 10² CFU, two bottles. It is of note that all bottles failing to return a positive result by SIFT-MS also failed to return a positive result by the conventional blood culture system, indicating that bacterial growth may have been absent from these bottles.

TABLE 7
VCs produced at statistically significant levels at 24 hours by five
bacteria under aerobic and anerobic growth conditions

| | 24 hrs 100 CFU | | 24 hrs 5 CFU | | | |
|---|---|---|---|---|---|---|
| Aerobic | | | | | | |
| S. pneumoniae | Acetaldehyde | 2515 | <0.001 | | | |
| | Acetic acid | 768 | 0.002 | | | |
| | Ethanol | 5012 | 0.021 | | | |
| | Pentanols | 285 | 0.018 | | | |
| | Acetone | 4333 | 0.001 | | | |
| | Dimethyl disulphide | 585 | 0.018 | | | |
| | Trimethylamine | 196 | 0.033 | | | |
| | Indole | 117 | 0.031 | | | |
| | 2 aminoacetophenone | 236 | 0.042 | | | |
| | Mass 105 | 2003 | <0.001 | | | |
| | Mass 121 | 149 | 0.029 | | | |
| | Mass 139 | 135 | 0.048 | | | |
| Ps. aeruginosa | — | | | — | | |
| E. coli | Formaldehyde | 124 | 0.018 | | | |
| | acetaldehyde | 15113 | 0.005 | | | |
| | Acetic acid | 5131 | 0.031 | | | |
| | Ethanol | 71575 | 0.02 | | | |
| | Pentanols | 1002 | 0.019 | | | |
| | Acetone | 2458 | 0.009 | | | |
| | Ammonia | 293 | 0.024 | Ammonia | 208 | 0.002 |
| | Methanethiol | 1819 | 0.003 | | | |
| | Dimethyl sulphide | 62 | 0.005 | | | |
| | Dimethyl disulphide | 646 | 0.03 | | | |
| | Indole | 497 | 0.03 | | | |
| | Propene | 1397 | 0.022 | | | |
| | Mass 101 | 447 | 0.049 | | | |
| | Mass 105 | 2084 | 0.049 | | | |
| | Mass 121 | 556 | 0.027 | | | |
| | Mass 155 | 256 | 0.026 | | | |
| S. aureus | Formaldehyde | 120 | 0.012 | Formaldehyde | 138 | <0.001 |
| | Acetaldehyde | 2887 | 0.008 | acetaldehyde | 3072 | 0.018 |
| | Acetic acid | 1002 | 0.006 | | | |
| | Ethanol | 21710 | 0.019 | | | |
| | Pentanols | 129 | 0.022 | Pentanols | 131 | 0.015 |
| | Acetone | 1673 | 0.003 | Acetone | 1518 | 0.009 |
| | Ammonia | 191 | 0.027 | Methanethiol | 608 | 0.007 |
| | Dimethyl disulphide | 226 | 0.017 | Dimethyl | 241 | 0.036 |
| | | | | disulphide | | |
| | Trimethylamine | 123 | 0.01 | Trimethylamine | 99 | 0.023 |
| | Indole | 90 | 0.01 | Indole | 69 | 0.049 |
| | 2 aminoacetophenone | 414 | 0.047 | | | |
| | Propene | 1859 | 0.014 | Propene | 1895 | 0.008 |
| | Mass 105 | 980 | 0.022 | Mass 105 | 963 | 0.005 |
| | Mass 121 | 271 | 0.042 | Mass 121 | 267 | 0.047 |
| | Mass 139 | 339 | 0.031 | | | |
| N. meningitidis | — | | | — | | |
| Anaerobic | | | | | | |
| S. pneumoniae | Acetaldehyde | 550 | 0.008 | | | |
| | Ethanol | 4623 | 0.007 | Ethanol | 4630 | 0.041 |
| | Acteone | 1564 | 0.01 | Acteone | 1437 | 0.001 |
| | Hydrogen sulphide | 4511 | 0.013 | Hydrogen | 4223 | 0.019 |
| | | | | sulphide | | |
| | Methanethiol | 726 | <0.001 | Methanethiol | 597 | 0.012 |
| | Dimethyldisulphide | 313 | 0.03 | Dimethyldisulphide | 303 | 0.008 |
| | 2 | 430 | 0.03 | | | |
| | aminoacetophenone | | | | | |
| | Mass 105 | 154 | 0.04 | | | |
| Ps. aeruginosa | — | | | — | | |
| E. coli | Acetaldehyde | 39964 | 0.016 | Acetaldehyde | 27529 | 0.018 |
| | | | 0.005 | Acetic acid | 40001 | 0.033 |
| | Ethanol | 210055 | 0.005 | Ethanol | 178025 | 0.007 |
| | Pentanols | 1352 | 0.011 | Pentanols | 780 | 0.031 |
| | | | | Ammonia | 6079 | 0.022 |
| | Acetone | 1284 | 0.004 | Acetone | 939 | 0.022 |
| | Hydrogen sulphide | 478509 | 0.032 | Hydrogen | 351221 | 0.024 |
| | | | | sulphide | | |
| | Methanethiol | 10206 | 0.013 | Methanethiol | 5752 | 0.006 |
| | | | | Dimethylsulphide | 757 | 0.047 |
| | Indole | 3174 | 0.047 | Indole | 1561 | 0.021 |
| | 2aminoacetophenone | 41907 | 0.046 | 2aminoacetophenone | 18488 | 0.002 |
| | Propene | 20751 | 0.006 | Propene | 15088 | 0.01 |
| | | | | Mass 101 | 6528 | 0.017 |
| | Mass 105 | 8691 | 0.032 | Mass 105 | 4916 | 0.01 |
| | Mass 121 | 2693 | 0.029 | Mass 121 | 2023 | 0.018 |
| | Mass 139 | 45810 | 0.04 | Mass 139 | 19866 | 0.006 |
| | | 14115 | | | | |
| S. aureus | Acetone | 737 | 0.044 | | | |
| | Ammonia | 3279 | 0.027 | | | |
| N. meningitidis | Acetic acid | 1730 | 0.006 | Acetic acid | 1817 | 0.043 |
| | Ammonia | 800 | 0.003 | Ammonia | 955 | 0.003 |
| | Hydrogen sulphide | 363 | 0.012 | | | |
| | Methanethiol | 339 | 0.011 | | | |
| | Mass 139 | 111 | 0.019 | | | |

### The Effects of Antibiotics on Bacterial Blood Culture VC Production

Approximately 10³ CFU E. coli or S. aureus were inoculated into Biomerieux BacT/ALERT® SA bottles including 9 ml of fresh uninfected blood. Each bacterial species was incubated in triplicate either alone or in the presence of antibiotics above or below their predetermined minimal inhibitory concentrations (MIC).

Gentamicin was added to E. coli cultures at either 2 μg/ml or 0.05 μg/ml (MIC 0.25-1 μg/m)l. Flucloxacillin was added to S. aureus cultures at 2 μg/ml or 0.05 μg/ml (MIC 0.12-0.5 μg/ml).

### Bacterial VC at 6 Hours in BacT/ALERT®FA Aerobic Medium

The mean concentration and range (ppb v/v) of each SIMscan VC analyte is recorded in Table 1 (above) for each species and uninoculated controls following six hours incubation in BacT/ALERT®FA medium-containing bottles. The patterns of high, medium and low SIMscan analytes defined by the relative concentrations of each VC, compared to each other, differed for each of the five test species.

Table 1 illustrates the characteristic VC patterns for each test species. For example, the headspaces of Pseudomonas aeruginosa cultures had relatively high absolute concentrations of acetic acid and acetone, compared to other analytes (p<0.001), and an absence of methanethiol. Streptococcus pneumoniae metabolic VCs were marked by high acetaldehyde, acetone, ethanol and dimethyl sulfide compared with intermediate acetic acid, ammonia and dimethyldisulfide (p<0.001). The dimethyldisulfide concentration (180 ppb v/v) significantly exceed low hydrogen sulfide and methanethiol (p<0.001). High relative concentrations of acetaldehyde, ethanol and dimethyl sulfide significantly exceeded intermediate acetic acid, acetone and hydrogen sulfide (p<0.001). While hydrogen sulfide, the lowest intermediate concentration analyte, differed significantly from lower ammonia, methanethiol and dimethyldisulfide (p<0.001) in Escherichia coli cultures. Relatively high concentrations of ethanol and acetone exceeded intermediate concentrations of acetaldehyde, acetic acid, ammonia and dimethyl sulfide (p<0.001) in cultures of Staphylococcus aureus. These intermediate concentration analytes were all significantly higher than the concentrations of hydrogen sulfide, methanethiol and dimethyldisulfide (p<0.001). At six hours, Neisseria meningitidis cultures demonstrated very high acetone production compared with low acetaldehyde, acetic acid, ethanol dimethyl sulfide and dimethyldisulfide (p<0.001) and no detectable hydrogen sulfide or methanethiol.

The effects of antibiotics on bacterial blood culture VC production

SIMscan VC concentrations were measured in triplicate E. coli or S. aureus test and control culture headspaces following six or 22 hours of incubation in the presence or absence of gentamicin or flucloxicillin above or below their MIC. The VC analyte concentrations of uninoculated control media (containing blood) did not change significantly (<20% for any analyte) from background levels between six and 22 hour measurements. The mean, minima and maxima integrated headspace analyte concentration measurements obtained from E. coli and S. aureus cultures are compared in Tables 8 and 9.

After six hours of incubation the production of pentanols and hydrogen sulfide indicated growth of E. coli. The production of hydrogen sulfide was eliminated by gentamicin and a concentration-dependent relationship between the antibiotic concentrations was not, therefore, apparent at six hours. The VCs recorded in Table 8 also demonstrate growth of E. coli at 22 hours as well as the inhibitory effects of gentamicin. Those VCs consistent with concentration-dependent discrimination (p≦0.005) between the metabolic inhibitory effect of gentamicin at concentrations above or below its MIC for this organism include acetic acid, 2-aminoacetophenone, dimethyldisulfide, ethanol, hydrogen sulfide, methanethiol, pentanols and propanol.

TABLE 8
Effect of gentamicin on E. coli VC production

| | E. coli 6 h | | | | | |
|---|---|---|---|---|---|---|
| VC | Control | | >MIC | | <MIC | |
| | Mean concentration, ppb (range) | | | | | |
| Hydrogen sulfide | 50 | (30-50) | 0 | | 0 | |
| Pentanols | 110 | (90-130) | 90 | (70-190) | 120 | (80-120) |
| | E. coli 22 h | | | | | |
| | Control | | >MIC | | <MIC | |
| | Mean concentration, ppb (range) | | | | | |
| Acetaldehyde | 7000 | (5700-7700) | 2700 | (1200-4700) | 3300 | (3000-3600) |
| Acetic acid | 4500 | (3600-5000) | 600 | (480-700) | 1950 | (1800-2100) |
| Aminoacetophenone | 3400 | (2400-3900) | 280 | (200-560) | 900 | (800-1000) |
| DMDS | 700 | (600-750) | 190 | (170-220) | 380 | (320-400) |
| DMS | 220 | (180-250) | 30 | (25-30) | 30 | (10-60) |
| Ethanol | 76000 | (70000-79000) | 16000 | (10000-37000) | 42000 | (40000-44000) |
| Formaldehyde | 260 | (250-260) | 110 | (20-200) | 140 | (120-170) |
| Hydrogen sulfide | 830 | (800-850) | 6 | (5-13) | 1030 | (900-1100) |
| Indole | 690 | (550-760) | 110 | (70-150) | 210 | (170-240) |
| Methanethiol | 1600 | (1600-1600) | 150 | (110-300) | 1400 | (1000-1700) |
| Pentanols | 390 | (280-440) | 90 | (60-130) | 220 | (180-270) |
| Propanol | 13000 | (11000-14000) | 1700 | (800-3100) | 5800 | (5700-6000) |

S. aureus growth at six hours of incubation is indicated in Table 9 by the production of ammonia and dimethylsulfide. Production of these analytes was inhibited to uninoculated, antibiotic-containing control levels by flucloxacillin above and below its MIC for this organism. After 22 hours of in vitro blood culture acetaldehyde, 2-aminoacetophenone, ethanol, formaldehyde and indol concentrations above uninoculated controls indicated growth. Those VCs that discriminated between the metabolic effects of flucloxacillin concentrations in a concentration-dependent manner (p≦0.05), recorded in Table 9, include 2-aminoacetophenone, ethanol and formaldehyde.

TABLE 9
Effect of flucloxacillin on S. aureus VC production

| | S. aureus 6 h | | | | | |
|---|---|---|---|---|---|---|
| VC | Control | | >MIC | | <MIC | |
| | Mean concentration, ppb (range) | | | | | |
| Ammonia | 200 | (150-300) | 50 | (20-80) | 40 | (10-80) |
| DMS | 80 | (60-110) | 10 | (0-20) | 30 | (10-40) |
| | S. aureus 22 h | | | | | |
| | Control | | >MIC | | <MIC | |
| | Mean concentration, ppb (range) | | | | | |
| Aminoacetophenone | 210 | (200-210) | 60 | (25-80) | 200 | (180-210) |
| Ethanol | 7700 | (7200-8100) | 1900 | (1500-2100) | 7500 | (7400-7700) |
| Formaldehyde | 70 | (50-80) | 20 | (10-30) | 60 | (50-65) |

This SIFT-MS study illustrates three new, significant findings. First, the growth of less than 10 colony forming units of five bacterial species can be detected by SIFT-MS analysis of headspace VCs at six hours using standard Biomerieux BacT/ALERT®FA aerobic blood culture. It also shows that the VC profiles discriminated between seeded aerobic blood cultures of different species as early as six hours of culture in both BacT/ALERT® SA and FA media. There were significant distinctions between the relative concentrations of analytes for each of the test species and these VC concentration patterns differed markedly between the species. For example the concentrations of acetaldehyde, acetic acid ammonia, ethanol and dimethyl sulfide clearly distinguished Staphylococcus aureus from the other species. High acetone and undetectable hydrogen sulfide and methanethiol characterized Neisseria meningitidis cultures while high acetic acid and acetone unaccompanied by high ethanol, acetaldehyde, dimethyl disulfide or dimethyl sulfide marked Pseudomonas aeruginosa growth from Esherichia coli and Streptococcus pneumoniae. Although a direct comparison of the absolute concentrations of headspace VC analytes between cultured species may not be warranted, due to undefined differences in species substrate utilization, culture lag phase, bacterial growth and metabolic efficiency, the relative abundance of VCs for a particular species demonstrates species-specific patterns.

In addition to the different VC patterns between species, the concentration-dependent effects of antibiotics above and below their MIC were demonstrated by significant changes to the VC profiles of the two test micro-organisms, E. coli and S. aureus. For example, at six hours the production of hydrogen sulfide by E. coli was eliminated and the production of all headspace VCs (except formaldehyde) was significantly inhibited (p<0.005) at 22 hours by gentamicin above its demonstrated MIC (Table 8). Significant (p<0.01) reductions in most analytes (except hydrogen sulfide and methanethiol) also resulted from incubation with gentamicin below its MIC but these concentrations generally exceeded the corresponding values at the higher antibiotic concentration.

Flucloxacillin, above and below its demonstrated MIC, significantly reduced (p<0.01) the production of ammonia and dimethylsulfide by S. aureus at six hours incubation. The inhibition of aminoacetophenone, ethanol and formaldehyde was notable (p<0.01) at 22 hours with Flucloxacillin above its MIC while the lower antibiotic concentration of was not inhibitory (Table 9).

It is also notable that the SIMscan measurement of VC concentrations from each bacterial culture headspace was completed and recorded within 30 seconds; without any sample preparation.

Consequently it is possible to formulate a sampling and testing algorithm capable of establishing the presence, the likely identification of micro-organisms and their in vitro susceptibility to antimicrobial agents.

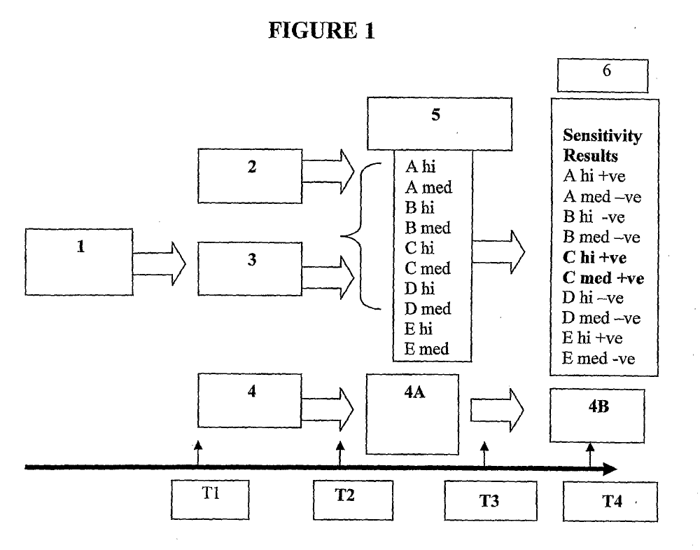

FIG. 1 illustrates a schematic form of the steps of the present invention which are involved in the detection and determination of the presence of micro-organisms in a blood culture using SIFT-MS to analyse the VCs in the headspace above the blood culture under investigation.

To utilise the procedure a sample of blood, for instance 10 ml, is taken from the patient and is injected into the culture bottle 1 to form the primary culture. Preferably the bottle has a piercable septum top and in a highly preferred form the bottle may be that known under the trade name Biomerieux BacTALERT®. When the micro-organisms in the blood sample have had time to multiply as indicated at point T1, which may typically be three hours from the start of the test, the primary culture is divided into secondary bottles 2, 3 and 4. While in the form illustrated the primary culture is divided into three secondary BacT/ALERT® bottles, it will be understood the number of divisions of the primary culture will be at the option of the operator and of the circumstances.

At the expiry of a predetermined time, which may be a further three hours from the start of the test at point T2 illustrated in FIG. 1, the secondary cultures are tested by SIFT-MS for growth of micro-organisms. If the test is positive, a report can be provided that the patient has a blood infection. The blood culture in the secondary bottle 4 is retained as the control culture.

The blood cultures are split into small quantities as indicated at 5, for instance 5 ml aliquots which are separately put into bottles which contain high and medium concentrations of different antimicrobial agents as illustrated as A through E. At point T3 which is typically 22 hours from the start of the test, the VCs are tested by SIFT-MS to identify whether the micro-organisms have grown in the presence of each antimicrobial agent and the susceptibility of the micro-organisms to the antimicrobial agent.

As indicated in FIG. 1, at this point the tertiary blood culture identified as C indicates a positive sensitivity to both a medium concentration and a high concentration of the antimicrobial agent.

It is possible at this stage to identify the infective micro-organism from its VC profile and determine the resistance and susceptibility of the micro-organisms to the antimicrobial agent. Because of the different antimicrobial agents and the different levels of agent in each bottle, not only can the type of micro-organism be readily identified and diagnosed, but also the type of antimicrobial agent and the optimum level of the agent be identified at point T4 which is typically within a 24 hour time span. Consequently by using high and medium concentrations of each antimicrobial agent, additional information can be ascertained as to the likely dose of antimiriobial agent that might be used to treat the patient.

It will be understood that while specific times are recited for the implementation of the various steps at points T1, T2, T3 and T4, these times can vary dependent upon the circumstances and the requirements of the operator.

Having described preferred methods of putting the invention into effect, it will be apparent to those skilled in the art to which this invention relates, that modifications and amendments to various features and items can be effected and yet still come within the general concept of the invention. It is to be understood that all such modifications and amendments are intended to be included within the scope of the present invention.

1. A method of determining the metabolic consequences of an antimicrobial agent for micro-organisms comprising:
establishing by SIFT-MS the presence of a micro-organism(s) by sampling the volatile or semi-volatile compounds (VCs) produced by the micro-organism(s);
identifying micro-organism(s);
analysing the VCs produced by the micro-organism(s); and
determining the antimicrobial susceptibility of the micro-organism.

2. A method of identifying a micro-organism and the metabolic consequences of an antimicrobial agent on the micro-organism comprising the steps of:
inserting a sample containing the micro-organisms into a primary culture container;
allowing micro-organisms within the primary culture of the sample to multiply in the container;
dividing the primary culture into a plurality of secondary cultures and charging separate containers with individual secondary cultures;
analysing the VCs in the headspace above the secondary cultures by means of SIFT-MS to ascertain whether micro-organisms exist in the secondary culture;
determining the type of micro-organism;
splitting the secondary cultures into a plurality of tertiary cultures and inserting the tertiary cultures into separate containers with each container including a specific antimicrobial agent at a specific concentration; and
analysing the VCs in the headspace above each tertiary culture to determine the antimicrobial susceptibility of the micro-organism to each antimicrobial agent at each concentration.

3. The method as claimed in claim 2, wherein the primary culture is divided into the secondary cultures after a period of rest or incubation from the commencement of the test.

4. The method as claimed in claim 2, wherein the primary culture is divided into a plurality of secondary cultures with one of the secondary cultures being retained as a control culture to enable a positive or negative report to be generated as to the presence of micro-organism(s) and to enable the micro-organism(s) to be identified.

5. The method as claimed in claim 2, wherein the primary culture is divided into three secondary cultures.

6. The method as claimed in claim 2, wherein the secondary culture is tested after a period of rest from the commencement of the test and if the test provides a positive result, the secondary culture is divided into the plurality of tertiary cultures.

8. The method as claimed in claim 2, wherein the secondary culture is divided into a plurality of tertiary cultures which are each combined with a specific antimicrobial agent at a specific concentration.

9. The method as claimed in claim 2, wherein the tertiary cultures are tested after a period of rest from the commencement of the test to determine the susceptibility of the micro-organism to the antimicrobial agent.

10. The method as claimed in claim 1, wherein the susceptibility of the micro-organism to the antimicrobial agent is determined by the presence of a specific concentration of the chosen antimicrobial agent.

11. A method of identifying a micro-organism and the metabolic consequences of an antimicrobial agent in blood culture comprising the steps of:
inserting a primary blood culture into a container;
allowing micro-organisms within the primary blood culture to multiply in the container;
dividing the culture into a plurality of secondary cultures and charging separate containers with individual secondary cultures;
analysing the VCs in the headspace above the secondary cultures by means of SIFT-MS to ascertain whether micro-organisms are present in the secondary blood culture and to determine the identity of the micro-organisms;
splitting the secondary cultures into a plurality of tertiary cultures and inserting the tertiary cultures into separate containers with each container including a specific antimicrobial agent at a specific concentration; and
analysing the VCs in the headspace above each tertiary culture to determine whether the micro-organisms in the tertiary culture have grown and to ascertain whether the antimicrobial agent has been inhibitory to the growth of the micro-organisms.

12. The method as claimed in claim 1, wherein a report is generated in which the micro-organism is identified and the antimicrobial susceptibility of the bacterium or micro-organism is displayed.
